SEQUENCE LISTING (13 sequences)

SEQ ID NO: 1 - protein, 418 aa, unidentified
  source 1..418: note = photosynthetic microorganism
MGYKKIQVPAVGDKITVNADHSLNVPDNPIIPFIEGDGIGVDVSPVMIKVVDAAVEKAYGGKRKISWMEVYAGEKATQVYDQDTWLPQETLDAVKDYVVSIKGPLTTPVGGGIRSLNVALRQQLDLYVCLRPVVWFEGVPSPVKKPGDVDMVIFRENSEDIYAGIEWKAGSPEATKVIKFLKEEMGVTKIRFDQDCGIGIKPVSKEGTKRLVRKALQYVVDNDRKSLTIVHKGNIMKFTEGAFKDWGYEVAKEEFGAELLDGGPWMKFKNPKTGREVVVKDAIADAMLQQILLRPAEYDVIATLNLNGDYLSDALAAEVGGIGIAPGANLSDTVAMFEATHGTAPKYAGKDQVNPGSVILSAEMMLRHLGWTEAADLIIKGTNGAIKAKTVTYDFERLMEGATLVSSSGFGEALIKHM

SEQ ID NO: 2 - protein, 1257 aa, unidentified
ATGGGTTACAAGAAGATTCAGGTTCCAGCCGTCGGCGACAAAATCACCGTCAACGCAGACCATTCTCTCAATGTCCCTGATAACCCGATCATTCCCTTCATCGAAGGTGACGGCATTGGCGTCGACGTCAGCCCTGTGATGATCAAAGTGGTTGATGCTGCCGTAGAGAAAGCCTACGGGGGTAAGCGCAAGATTTCCTGGATGGAGGTTTATGCTGGCGAAAAAGCAACTCAGGTCTATGACCAGGACACCTGGCTGCCCCAGGAAACCCTGGACGCGGTCAAGGATTACGTGGTCTCCATCAAAGGCCCGCTGACCACTCCGGTCGGTGGCGGCATCCGTTCCCTCAACGTCGCCCTGCGCCAACAGCTCGATCTCTATGTCTGCCTTCGCCCTGTGGTGTGGTTCGAAGGTGTGCCGAGCCCGGTGAAAAAGCCTGGCGACGTCGACATGGTGATCTTCCGCGAGAACTCCGAAGACATTTATGCCGGTATCGAATGGAAAGCCGGCTCCCCTGAGGCCACCAAGGTCATCAAATTCCTGAAAGAAGAAATGGGCGTCACCAAGATCCGTTTCGACCAGGATTGCGGCATCGGCATCAAGCCGGTTTCCAAAGAAGGCACCAAGCGTCTGGTGCGCAAGGCGCTGCAATACGTGGTGGACAACGACCGCAAGTCGCTGACCATCGTGCACAAGGGCAACATCATGAAATTCACCGAAGGTGCCTTCAAGGACTGGGGCTACGAGGTGGCGAAGGAAGAATTCGGCGCCGAGCTGCTCGATGGCGGCCCATGGATGAAATTCAAGAACCCGAAAACCGGCCGCGAAGTCGTCGTCAAGGACGCCATCGCCGACGCCATGCTCCAGCAGATCCTGCTGCGTCCGGCCGAATACGATGTGATCGCCACCCTCAACCTCAACGGTGACTACCTGTCCGACGCCCTGGCGGCGGAAGTGGGCGGTATCGGTATCGCGCCGGGTGCCAACCTGTCCGACACCGTAGCCATGTTCGAGGCGACCCACGGTACTGCGCCGAAATATGCCGGCAAGGACCAGGTCAACCCGGGTTCGGTGATTTTGTCGGCGGAAATGATGCTGCGCCACCTGGGCTGGACCGAGGCGGCCGACCTGATCATCAAGGGCACCAATGGCGCCATCAAGGCCAAGACCGTGACCTACGACTTCGAACGTCTGATGGAAGGCGCCACACTGGTGTCTTCTTCGGGCTTCGGTGAAGCGCTGATCAAGCACATGTAA

SEQ ID NO: 3 - protein, 475 aa, unidentified
  source 1..475: note = photosynthetic microorganism
MYEKIQPPSEGSKIRFEAGKPIVPDNPIIPFIRGDGTGVDIWPATERVLDAAVAKAYGGQRKITWFKVYAGDEACDLYGTYQYLPEDTLTAIREYGVAIKGPLTTPIGGGIRSLNVALRQIFDLYACVRPCRYYTGTPSPHRTPEQLDVVVYRENTEDIYLGIEWKQGDPTGDRLIKLLNEDFIPNSPSLGKKQIRLDSGIGIKPISKTGSQRLIRRAIEHALRLEGRKRHVTLVHKGNIMKFTEGAFRDWGYELATTEFRTDCVTERESWILANQESKPDLSLEDNARLIEPGYDAMTPEKQAAVVAEVKAVLDSIGATHGNGQWKSKVLVDDRIADSIFQQIQTRPGEYSVLATMNLNGDYISDAAAAVVGGLGMAPGANIGDEAAIFEATHGTAPKHAGLDRINPGSVILSGVMMLEYLGWQEAADLITKGISQAIANREVTYDLARLMEPAVDQPLKCSEFAEAIVKHFDD

SEQ ID NO: 4 - protein, 1450 aa, unidentified
  source 1..1450: note = photosynthetic microorganism
AGGGCAAGCTTATGTACGAGAAGATTCAACCCCCTAGCGAAGGCAGCAAAATTCGCTTTGAAGCCGGCAAGCCGATCGTTCCCGACAACCCGATCATTCCCTTCATTCGTGGTGACGGCACTGGCGTTGATATCTGGCCCGCAACTGAGCGCGTTCTCGATGCCGCTGTCGCTAAAGCCTATGGCGGTCAGCGCAAAATCACTTGGTTCAAAGTCTACGCGGGTGATGAAGCCTGCGACCTCTACGGCACCTACCAATATCTGCCTGAAGATACGCTGACAGCGATCCGCGAGTACGGCGTGGCAATCAAAGGCCCGCTGACGACGCCGATCGGTGGTGGCATTCGATCGCTGAACGTGGCGCTACGGCAAATCTTCGATCTCTATGCCTGCGTCCGCCCCTGTCGCTACTACACCGGCACACCCTCGCCCCACCGCACGCCCGAACAACTCGATGTGGTGGTCTACCGCGAAAACACCGAGGATATCTACCTCGGCATCGAATGGAAGCAAGGTGATCCCACCGGCGATCGCCTGATCAAGCTGCTGAACGAGGACTTCATTCCCAACAGCCCCAGCTTGGGTAAAAAGCAAATCCGTTTGGATTCCGGCATTGGTATTAAGCCGATCAGTAAAACGGGTAGCCAGCGTCTGATTCGTCGTGCGATCGAGCATGCCCTACGCCTCGAAGGCCGCAAGCGACATGTCACCCTTGTCCACAAGGGCAACATCATGAAGTTCACGGAAGGTGCTTTCCGGGACTGGGGCTATGAACTGGCCACGACTGAGTTCCGAACCGACTGTGTGACTGAACGGGAGAGCTGGATTCTTGCCAACCAAGAAAGCAAGCCGGATCTCAGCTTGGAAGACAATGCGCGGCTCATCGAACCTGGCTACGACGCGATGACGCCCGAAAAGCAGGCAGCAGTGGTGGCTGAAGTGAAAGCTGTGCTCGACAGCATCGGCGCCACCCACGGCAACGGTCAGTGGAAGTCTAAGGTGCTGGTTGACGATCGCATTGCTGACAGCATCTTCCAGCAGATTCAAACCCGCCCGGGTGAATACTCGGTGCTGGCGACGATGAACCTCAATGGCGACTACATCTCTGATGCAGCGGCGGCGGTGGTCGGTGGCCTGGGCATGGCCCCCGGTGCCAACATTGGCGACGAAGCGGCGATCTTTGAAGCGACCCACGGCACAGCGCCCAAGCACGCTGGCCTCGATCGCATTAACCCCGGCTCGGTCATCCTCTCCGGCGTGATGATGCTGGAGTACCTAGGCTGGCAAGAGGCTGCTGACTTGATCACCAAGGGCATCAGCCAAGCGATCGCTAACCGTGAGGTCACCTACGATCTGGCTCGGTTGATGGAACCGGCGGTTGATCAACCACTCAAGTGCTCGGAATTTGCCGAAGCCATCGTCAAGCATTTCGACGATTAGGGATCCAGCGC

SEQ ID NO: 5 - protein, 1628 aa, unidentified
  source 1..1628: note = photosynthetic microorganism
MAFFTAASKADFQHQLQAALAQHISEQALPQVALFAEQFFGIISLDELTQRRLSDLAGCTLSAWRLLERFDHAQPQVRVYNPDYERHGWQSTHTAVEVLHHDLPFLVDSVRTELNRRGYSIHTLQTTVLSVRRGSKGELLEILPKGTTGEGVLHESLMYLEIDRCANAAELNVLSKELEQVLGEVRVAVSDFEPMKAKVQEILTKLDNSAFAVDADEKNEIKSFLEWLVGNHFTFLGYEEFTVVDQADGGHIEYDQNSFLGLTKMLRTGLTNEDRHIEDYAVKYLREPTLLSFAKAAHPSRVHRPAYPDYVSIREIDADGKVIKEHRFMGLYTSSVYGESVRVIPFIRRKVEEIERRSGFQAKAHLGKELAQVLEVLPRDDLFQTPVDELFSTVMSIVQIQERNKIRVFLRKDPYGRFCYCLAYVPRDIYSTEVRQKIQQVLMERLKASDCEFWTFFSESVLARVQLILRVDPKNRIDIDPLQLENEVIQACRSWQDDYAALTVETFGEANGTNVLADFPKGFPAGYRERFAAHSAVVDMQHLLNLSEKKPLAMSFYQPLASGPRELHCKLYHADTPLALSDVLPILENLGLRVLGEFPYRLRHTNGREFWIHDFAFTAAEGLDLDIQQLNDTLQDAFVHIVRGDAENDAFNRLVLTAGLPWRDVALLRAYARYLKQIRLGFDLGYIASTLNNHTDIARELTRLFKTRFYLARKLGSEDLDDKQQRLEQAILTALDDVQVLNEDRILRRYLDLIKATLRTNFYQTDANGQNKSYFSFKFNPHLIPELPKPVPKFEIFVYSPRVEGVHLRFGNVARGGLRWSDREEDFRTEVLGLVKAQQVKNSVIVPVGAKGGFLPRRLPLGGSRDEIAAEGIACYRIFISGLLDITDNLKDGKLVPPANVVRHDDDDPYLVVAADKGTATFSDIANGIAIDYGFWLGDAFASGGSAGYDHKKMGITAKGAWVGVQRHFRERGINVQEDSITVVGVGDMAGDVFGNGLLMSDKLQLVAAFNHLHIFIDPNPNPATSFAERQRMFDLPRSAWSDYDTSIMSEGGGIFSRSAKSIAISPQMKERFDIQADKLTPTELLNALLKAPVDLLWNGGIGTYVKASTESHADVGDKANDALRVNGNELRCKVVGEGGNLGMTQLGRVEFGLNGGGSNTDFIDNAGGVDCSDHEVNIKILLNEVVQAGDMTDKQRNQLLASMTDEVGGLVLGNNYKQTQALSLAARRAYARIAEYKRLMSDLEGRGKLDRAIEFLPTEEQLAERVAEGHGLTRPELSVLISYSKIDLKEQLLGSLVPDDDYLTRDMETAFPPTLVSKFSEAMRRHRLKREIVSTQIANDLVNHMGITFVQRLKESTGMTPANVAGAYVIVRDIFHLPHWFRQIEALDYQVSADVQLELMDELMRLGRRATRWFLRARRNEQNAARDVAHFGPHLKELGLKLDELLSGEIRENWQERYQAYVAAGVPELLARMVAGTTHLYTLLPIIEAADVTGQDPAEVAKAYFAVGSALDITWYISQISALPVENNWQALAREAFRDDVDWQQRAITIAVLQAGGGDSDVETRLALWMKQNDAMIERWRAMLVEIRAASGTDYAMYAVANRELNDVALSGQAVVPAAATAELELA

SEQ ID NO: 6 - protein, 4887 aa, unidentified
  source 1..4887: note = photosynthetic microorganism
ATGGCGTTCTTCACCGCAGCCAGCAAAGCCGACTTCCAGCACCAACTGCAAGCGGCACTGGCGCAGCACATCAGTGAACAGGCACTGCCACAAGTGGCGCTGTTCGCTGAACAATTCTTCGGCATCATTTCCCTGGACGAGCTGACCCAACGTCGCCTCTCCGACCTCGCTGGCTGTACTCTCTCTGCGTGGCGCCTGCTTGAGCGCTTCGATCACGCGCAACCGCAAGTGCGCGTCTACAACCCCGATTACGAACGTCACGGCTGGCAGTCGACCCACACCGCGGTCGAAGTGCTGCACCACGACTTGCCGTTCCTGGTGGACTCGGTGCGTACCGAGCTGAACCGTCGCGGCTACAGCATCCACACCCTGCAGACCACCGTGCTGAGCGTGCGTCGTGGCAGCAAGGGCGAATTGCTGGAAATCCTGCCAAAAGGCACCACCGGCGAAGGCGTTCTGCACGAGTCGCTGATGTACCTGGAAATCGACCGCTGCGCCAATGCGGCCGAATTGAATGTGCTGTCCAAGGAACTGGAGCAGGTCCTGGGTGAAGTCCGCGTCGCGGTCTCCGATTTCGAGCCGATGAAGGCCAAGGTGCAGGAAATCCTCACCAAGCTCGATAACAGCGCATTCGCCGTCGATGCCGACGAAAAGAATGAAATCAAGAGCTTCCTGGAATGGCTGGTGGGCAACCACTTCACCTTCCTCGGCTACGAAGAGTTCACCGTTGTCGATCAGGCCGATGGCGGCCACATCGAATACGACCAGAACTCCTTCCTCGGCCTGACCAAGATGCTGCGCACCGGTCTGACCAACGAAGACCGCCACATCGAAGACTATGCCGTGAAGTACCTGCGCGAACCGACACTGCTGTCGTTCGCCAAGGCGGCGCATCCGAGCCGCGTGCACCGTCCGGCCTACCCGGACTACGTGTCGATCCGCGAAATCGATGCCGACGGCAAAGTGATCAAGGAACACCGCTTCATGGGCCTGTACACCTCGTCGGTGTATGGCGAAAGCGTGCGTGTCATCCCGTTCATCCGCCGCAAGGTCGAGGAAATCGAGCGTCGCTCCGGCTTCCAGGCCAAGGCTCACCTGGGCAAGGAACTGGCTCAGGTTCTGGAAGTGCTGCCGCGTGACGATCTGTTCCAGACCCCGGTCGACGAACTGTTCAGCACCGTGATGTCGATCGTGCAGATCCAGGAACGCAACAAGATCCGCGTGTTCCTGCGTAAAGACCCGTACGGTCGTTTCTGCTACTGCCTGGCCTACGTGCCGCGTGACATCTACTCCACCGAAGTTCGCCAGAAGATCCAGCAAGTGCTGATGGAGCGCCTGAAAGCCTCCGACTGCGAATTCTGGACGTTCTTCTCCGAGTCCGTGCTGGCCCGCGTGCAACTGATCTTGCGCGTCGACCCGAAAAACCGCATCGACATCGACCCGCTGCAACTGGAAAACGAAGTGATCCAGGCCTGCCGCAGCTGGCAGGACGACTACGCTGCCCTGACCGTTGAAACCTTCGGCGAAGCCAACGGCACCAACGTGTTGGCCGACTTCCCGAAAGGCTTCCCGGCCGGCTACCGCGAGCGTTTCGCAGCGCATTCGGCCGTGGTCGACATGCAGCACTTGCTCAATCTGAGCGAGAAAAAGCCGCTGGCCATGAGCTTTTACCAGCCGCTGGCCTCCGGCCCACGCGAGCTGCACTGCAAGCTGTATCACGCCGATACCCCGCTGGCCCTGTCCGACGTGCTGCCGATCCTGGAAAACCTCGGCCTGCGCGTGCTGGGTGAGTTCCCGTACCGCCTGCGTCATACCAACGGCCGCGAGTTCTGGATCCACGACTTCGCGTTCACCGCTGCCGAAGGCCTGGACCTGGACATCCAGCAACTCAACGACACCCTGCAGGACGCGTTCGTCCACATCGTCCGTGGCGATGCCGAAAACGATGCGTTCAACCGTCTGGTGCTGACCGCCGGCCTGCCATGGCGCGACGTGGCGCTGCTGCGTGCCTACGCCCGCTACCTGAAGCAGATCCGCCTGGGCTTCGACCTCGGCTACATCGCCAGCACCCTGAACAACCACACCGACATCGCTCGCGAACTGACCCGGTTGTTCAAGACCCGTTTCTACCTGGCCCGCAAGCTGGGCAGCGAGGATCTGGACGACAAGCAACAGCGTCTGGAACAGGCCATCCTGACCGCGCTGGACGACGTTCAAGTCCTCAACGAAGACCGCATCCTGCGTCGTTACCTGGACCTGATCAAAGCAACCCTGCGCACCAACTTCTACCAGACCGACGCCAACGGCCAGAACAAGTCGTACTTCAGCTTCAAGTTCAACCCGCACTTGATTCCTGAACTGCCGAAACCGGTGCCGAAGTTCGAAATCTTCGTTTACTCGCCACGCGTCGAAGGCGTGCACCTGCGCTTCGGCAACGTTGCTCGTGGTGGTCTGCGCTGGTCGGACCGTGAAGAAGACTTCCGTACCGAAGTCCTCGGCCTGGTAAAAGCCCAGCAAGTGAAGAACTCGGTCATCGTGCCGGTGGGGGCGAAGGGCGGCTTCCTGCCGCGTCGCCTGCCACTGGGCGGCAGCCGTGACGAGATCGCGGCCGAGGGCATCGCCTGCTACCGCATCTTCATTTCGGGCCTGTTGGACATCACCGACAACCTGAAAGACGGCAAACTGGTACCGCCGGCCAACGTCGTGCGGCATGACGACGATGACCCGTACCTGGTGGTCGCGGCGGACAAGGGCACTGCAACCTTCTCCGACATCGCCAACGGCATCGCCATCGACTACGGCTTCTGGCTGGGTGACGCGTTCGCGTCCGGTGGTTCGGCCGGTTACGACCACAAGAAAATGGGCATCACCGCCAAGGGCGCGTGGGTCGGCGTACAGCGCCACTTCCGCGAGCGCGGCATCAATGTCCAGGAAGACAGCATCACGGTGGTCGGCGTGGGCGACATGGCCGGTGACGTGTTCGGTAACGGCCTGTTGATGTCTGACAAGCTGCAACTGGTTGCTGCGTTCAACCACCTGCACATCTTCATCGACCCGAACCCGAACCCGGCCACCAGCTTCGCCGAGCGTCAGCGCATGTTCGATCTGCCGCGCTCGGCCTGGTCCGACTACGACACCAGCATCATGTCCGAAGGCGGCGGCATCTTCTCGCGCAGCGCGAAGAGCATCGCCATCTCGCCACAGATGAAAGAGCGCTTCGACATCCAGGCCGACAAGCTGACCCCGACCGAACTGCTGAACGCCTTGCTCAAGGCGCCGGTGGATCTGCTGTGGAACGGCGGTATCGGTACCTACGTCAAAGCCAGCACCGAAAGTCACGCCGATGTCGGCGACAAGGCCAACGATGCGCTGCGCGTGAACGGCAACGAACTGCGCTGCAAAGTGGTGGGCGAGGGCGGTAACCTCGGCATGACCCAATTGGGTCGTGTGGAGTTCGGTCTCAATGGCGGCGGTTCCAACACCGACTTCATCGACAACGCCGGTGGCGTGGACTGCTCCGACCACGAAGTGAACATCAAGATCCTGCTGAACGAAGTGGTTCAGGCCGGCGACATGACCGACAAGCAACGCAACCAGTTGCTGGCGAGCATGACCGACGAAGTCGGTGGTCTGGTGCTGGGCAACAACTACAAGCAGACTCAGGCCCTGTCCCTGGCGGCCCGCCGTGCTTATGCGCGGATCGCCGAGTACAAGCGTCTGATGAGCGACCTGGAGGGCCGTGGCAAGCTGGATCGCGCCATCGAGTTCCTGCCGACCGAAGAGCAACTGGCCGAACGCGTTGCCGAAGGCCATGGCCTGACCCGTCCTGAGCTGTCGGTGCTGATCTCGTACAGCAAGATCGACCTCAAGGAGCAGCTGCTGGGCTCCCTGGTGCCGGACGACGACTACCTGACCCGCGACATGGAAACGGCGTTCCCGCCGACCCTGGTCAGCAAGTTCTCCGAAGCTATGCGTCGTCACCGCCTCAAGCGCGAGATCGTCAGCACCCAGATCGCCAACGATCTGGTCAACCACATGGGCATCACCTTCGTTCAGCGACTCAAAGAGTCCACGGGCATGACCCCGGCGAATGTTGCCGGTGCGTATGTGATTGTTCGGGATATCTTCCACCTCCCGCACTGGTTCCGTCAGATCGAAGCGCTGGACTACCAGGTTTCCGCTGACGTGCAGCTGGAGCTGATGGACGAGCTGATGCGTCTGGGCCGTCGCGCTACGCGCTGGTTCCTGCGTGCCCGTCGCAACGAGCAGAACGCTGCCCGTGACGTCGCGCATTTCGGTCCGCACCTCAAAGAGCTGGGCCTGAAGCTGGACGAGCTGCTGAGCGGCGAAATCCGCGAAAACTGGCAAGAGCGTTATCAGGCGTACGTCGCCGCCGGTGTTCCGGAACTGCTGGCGCGTATGGTGGCGGGGACGACCCACCTCTACACGCTGCTGCCGATCATCGAAGCGGCCGACGTGACCGGCCAGGATCCAGCCGAAGTGGCCAAGGCGTACTTCGCCGTGGGCAGCGCGCTGGACATCACCTGGTACATCTCGCAGATCAGCGCCTTGCCGGTTGAAAACAACTGGCAGGCCCTGGCCCGTGAAGCGTTCCGCGACGACGTCGACTGGCAGCAACGCGCGATTACCATCGCCGTTCTGCAAGCGGGTGGCGGTGATTCGGACGTGGAAACCCGTCTGGCACTGTGGATGAAGCAGAACGACGCCATGATCGAACGCTGGCGCGCCATGCTGGTGGAAATCCGTGCCGCCAGCGGCACCGACTACGCCATGTACGCGGTGGCCAACCGCGAGCTGAACGACGTGGCGCTGAGCGGTCAGGCAGTTGTGCCTGCTGCGGCGACTGCGGAGCTTGAGCTTGCTTGA

SEQ ID NO: 7-8 - skipped (2 entries)

SEQ ID NO: 9 - protein, 808 aa, unidentified
  source 1..808: note = photosynthetic microorganism
MYKLVQTIVNSDEKNVLGDFILELGKDHKRYFLRNEILQAFADYCHQFPKPAYFYHSSSLGTFIQYTHEIILDGENTWFVVRPKIASQEVWLLSADLTKFELMTPKALLDVSDRLVKRYQPHILEIDLHPFYSAAPRIDDSRNIGQGLTVLNHYFCNQALTDPEYWIDALFQSLKRLEYNGIKLLISNHIHSGLQLTKQIKLALEFVSHLSPQTPYIKFKFHLQELGLEPGWGNNAARVRETLELLERLMDNPEPAILETFVSRICAVFRVVLISIHGWVAQEDVLGRDETLGQVIYVLEQARSLENKMRAEIKLAGLDTLGIKPHIIILTRLIPNCEGTFCNLPLEKVDGTENAWILRVPFAESRPEITNNWISKFEIWPYLEKFALDAEAELLKQFQGKPNLIIGNYSDGNLVAFILSRKMKVTQCNIAHSLEKPKYLFSNLYWQDLEAQYHFSAQFTADLISMNAADFIITSSYQEIVGTPDTMGQYESYKCFTMPNLYHVIDGIDLFSPKFNVVLPGVSENIFFPYNQTTNRESHRRQHIQDLIFHQEHPEILGKLDHPHKKPIFSVSPITSIKNLTGLVECFGKSEELQKHSNLILLTSKLHPDLGTNSEEIQEIAKIHAIIDQYHLHHKIRWLGMRLPLRDIAETYRVIADFQGIYIHFALYESFSRSILEAMISGLPTFTTQFGGSLEIIENHDQGFNLNPTDLAGTAKTIINFLEKCENYPEHWLENSQWMIERIRHKYNWNSHTNQLLLLTKMFSFWNFIYPEDNEARDRYMESLFHLLYKPIADHILSEHLSKIRNHN

SEQ ID NO: 10 - protein, 2427 aa, unidentified
  source 1..2427: note = photosynthetic microorganism
ATGTATAAATTAGTGCAAACTATTGTTAACAGTGATGAAAAAAATGTTTTAGGTGACTTTATCTTAGAATTAGGCAAGGATCATAAACGTTACTTTTTAAGAAATGAGATTTTACAAGCTTTTGCAGATTATTGTCACCAATTCCCAAAACCCGCTTATTTTTATCACTCTTCCTCTTTAGGGACATTCATTCAATACACCCATGAAATAATTTTAGATGGTGAAAATACTTGGTTTGTAGTTAGACCAAAGATTGCGAGTCAAGAAGTATGGTTATTAAGCGCGGACTTGACTAAGTTTGAGTTAATGACACCGAAAGCATTATTAGATGTGAGCGATCGCTTAGTAAAGCGTTATCAACCGCACATTTTAGAAATTGATCTCCATCCCTTTTATTCAGCAGCACCAAGAATTGATGATTCCAGAAATATTGGCCAAGGTTTAACCGTTCTTAATCATTATTTTTGTAATCAAGCATTGACAGATCCTGAATATTGGATTGACGCATTATTTCAATCATTAAAAAGATTAGAATATAACGGCATCAAATTATTAATTAGTAATCATATTCATTCAGGTTTGCAACTAACAAAGCAAATCAAACTAGCGTTAGAATTTGTGAGTCATTTATCCCCCCAGACACCATATATAAAATTTAAATTTCATCTTCAAGAACTCGGTTTAGAACCAGGTTGGGGTAATAATGCAGCCAGAGTCAGAGAAACCTTAGAACTGCTGGAAAGACTCATGGATAATCCCGAACCTGCAATTTTAGAAACCTTTGTTTCTCGCATTTGTGCAGTTTTCCGCGTCGTCCTTATTTCCATCCATGGTTGGGTTGCACAAGAAGATGTTTTAGGCAGAGATGAAACATTAGGACAAGTTATTTATGTTTTAGAACAAGCCCGCAGTTTAGAAAATAAAATGCGGGCAGAAATTAAACTTGCAGGTTTAGATACATTAGGAATTAAACCCCATATCATTATATTAACTCGACTGATTCCCAATTGTGAAGGCACATTTTGTAACTTACCATTAGAAAAAGTTGATGGTACAGAAAATGCTTGGATTTTGCGCGTTCCTTTTGCAGAATCTCGACCGGAAATTACCAACAACTGGATTTCTAAATTTGAAATTTGGCCTTATTTAGAAAAATTTGCTCTTGATGCCGAAGCAGAACTTTTAAAACAATTCCAAGGAAAGCCCAATCTAATTATTGGTAACTACAGTGACGGGAACTTAGTTGCTTTTATTCTCTCCCGAAAAATGAAAGTTACCCAATGTAATATTGCCCATTCCCTCGAAAAACCTAAATATCTATTTAGTAACTTATATTGGCAAGATTTAGAAGCACAATATCACTTTTCTGCCCAATTTACCGCTGATTTAATCAGTATGAATGCCGCAGATTTTATTATCACATCATCCTATCAAGAAATTGTAGGTACACCAGATACAATGGGACAATATGAATCTTATAAATGTTTCACCATGCCCAACTTATATCATGTAATTGATGGCATTGATTTATTTAGCCCTAAATTCAATGTGGTATTACCAGGAGTCAGTGAAAATATATTTTTTCCCTACAACCAAACAACAAATAGAGAATCCCACCGTCGTCAACATATCCAAGACCTAATTTTCCATCAAGAACACCCAGAAATTCTCGGTAAATTAGATCATCCTCATAAAAAACCGATCTTTTCCGTTAGTCCCATTACCTCAATTAAAAACCTCACAGGTTTAGTTGAATGTTTCGGTAAAAGTGAAGAATTACAAAAACATAGTAACCTAATTTTATTAACCAGTAAACTTCATCCAGACTTAGGAACAAACTCCGAAGAAATTCAAGAAATAGCAAAAATTCATGCGATTATTGATCAATATCATCTTCACCATAAAATCCGCTGGTTGGGAATGCGTCTTCCTCTCCGCGATATTGCTGAAACCTATCGTGTAATTGCCGATTTTCAAGGGATTTATATTCACTTTGCCCTTTATGAATCCTTTAGCAGAAGTATTTTAGAAGCAATGATTTCTGGATTACCAACTTTTACAACTCAATTTGGTGGTTCATTAGAAATTATTGAAAACCATGATCAAGGATTTAACCTCAACCCCACAGACTTAGCAGGAACAGCCAAAACAATTATCAACTTCTTAGAAAAATGTGAAAATTATCCAGAACATTGGCTAGAAAATTCTCAATGGATGATTGAACGCATTCGCCATAAATATAACTGGAATTCCCACACAAATCAACTCCTGTTATTAACGAAAATGTTTAGCTTTTGGAACTTCATCTATCCCGAAGATAACGAAGCCAGAGATCGTTACATGGAAAGTTTATTTCATCTTCTTTATAAACCTATAGCTGACCATATTTTATCAGAACATCTAAGCAAAATCAGAAATCATAATTAA

SEQ ID NO: 11 - protein, 709 aa, unidentified
  source 1..709: note = photosynthetic microorganism
MAAQNLYILHIQTHGLLRGQNLELGRDADTGGQTKYVLELAQAQAKSPQVQQVDIITRQITDPRVSVGYSQAIEPFAPKGRIVRLPFGPKRYLRKELLWPHLYTFADAILQYLAQQKRTPTWIQAHYADAGQVGSLLSRWLNVPLIFTGHSLGRIKLKKLLEQDWPLEEIEAQFNIQQRIDAEEMTLTHADWIVASTQQEVEEQYRVYDRYNPERKLVIPPGVDTDRFRFQPLGDRGVVLQQELSRFLRDPEKPQILCLCRPAPRKNVPALVRAFGEHPWLRKKANLVLVLGSRQDINQMDRGSRQVFQEIFHLVDRYDLYGSVAYPKQHQADDVPEFYRLAAHSGGVFVNPALTEPFGLTILEAGSCGVPVVATHDGGPQEILKHCDFGTLVDVSRPANIATALATLLSDRDLWQCYHRNGIEKVPAHYSWDQHVNTLFERMETVALPRRRAVSFVRSRKRLIDAKRLVVSDIDNTLLGDRQGLENLMTYLDQYRDHFAFGIATGRRLDSAQEVLKEWGVPSPNFWVTSVGSEIHYGTDAEPDISWEKHINRNWNPQRIRAVMAQLPFLELQPEEDQTPFKVSFFVRDRHETVLREVRQHLRRHRLRLKSIYSHQEFLDILPLAASKGDAIRHLSLRWRIPLENILVAGDSGNDEEMLKGHNLGVVVGNYSPELEPLRSYERVYFAEGHYANGILEALKHYRFFEAIA

SEQ ID NO: 12 - protein, 2130 aa, unidentified
  source 1..2130: note = photosynthetic microorganism
GTGGCAGCTCAAAATCTCTACATTCTGCACATTCAGACCCATGGTCTGCTGCGAGGGCAGAACTTGGAACTGGGGCGAGATGCCGACACCGGCGGGCAGACCAAGTACGTCTTAGAACTGGCTCAAGCCCAAGCTAAATCCCCACAAGTCCAACAAGTCGACATCATCACCCGCCAAATCACCGACCCCCGCGTCAGTGTTGGTTACAGTCAGGCGATCGAACCCTTTGCGCCCAAAGGTCGGATTGTCCGTTTGCCTTTTGGCCCCAAACGCTACCTCCGTAAAGAGCTGCTTTGGCCCCATCTCTACACCTTTGCGGATGCAATTCTCCAATATCTGGCTCAGCAAAAGCGCACCCCGACTTGGATTCAGGCCCACTATGCTGATGCTGGCCAAGTGGGATCACTGCTGAGTCGCTGGTTGAATGTACCGCTAATTTTCACAGGGCATTCTCTGGGGCGGATCAAGCTAAAAAAGCTGTTGGAGCAAGACTGGCCGCTTGAGGAAATTGAAGCGCAATTCAATATTCAACAGCGAATTGATGCGGAGGAGATGACGCTCACTCATGCTGACTGGATTGTCGCCAGCACTCAGCAGGAAGTGGAGGAGCAATACCGCGTTTACGATCGCTACAACCCAGAGCGCAAGCTTGTCATTCCACCGGGTGTCGATACCGATCGCTTCAGGTTTCAGCCCTTGGGCGATCGCGGTGTTGTTCTCCAACAGGAACTGAGCCGCTTTCTGCGCGACCCAGAAAAACCTCAAATTCTCTGCCTCTGTCGCCCCGCACCTCGCAAAAATGTACCGGCGCTGGTGCGAGCCTTTGGCGAACATCCTTGGCTGCGCAAAAAAGCCAACCTTGTCTTAGTACTGGGCAGCCGCCAAGACATCAACCAGATGGATCGCGGCAGTCGGCAGGTGTTCCAAGAGATTTTCCATCTGGTCGATCGCTACGACCTCTACGGCAGCGTCGCCTATCCCAAACAGCATCAGGCTGATGATGTGCCGGAGTTCTATCGCCTAGCGGCTCATTCCGGCGGGGTATTCGTCAATCCGGCGCTGACCGAACCTTTTGGTTTGACAATTTTGGAGGCAGGAAGCTGCGGCGTGCCGGTGGTGGCAACCCATGATGGCGGCCCCCAGGAAATTCTCAAACACTGTGATTTCGGCACTTTAGTTGATGTCAGCCGACCCGCTAATATCGCGACTGCACTCGCCACCCTGCTGAGCGATCGCGATCTTTGGCAGTGCTATCACCGCAATGGCATTGAAAAAGTTCCCGCCCATTACAGCTGGGATCAACATGTCAATACCCTGTTTGAGCGCATGGAAACGGTGGCTTTGCCTCGTCGTCGTGCTGTCAGTTTCGTACGGAGTCGCAAACGCTTGATTGATGCCAAACGCCTTGTCGTTAGTGACATCGACAACACACTGTTGGGCGATCGTCAAGGACTCGAGAATTTAATGACCTATCTCGATCAGTATCGCGATCATTTTGCCTTTGGAATTGCCACGGGGCGTCGCCTAGACTCTGCCCAAGAAGTCTTGAAAGAGTGGGGCGTTCCTTCGCCAAACTTCTGGGTGACTTCCGTCGGCAGCGAGATTCACTATGGCACCGATGCTGAACCGGATATCAGCTGGGAAAAGCATATCAATCGCAACTGGAATCCTCAGCGAATTCGGGCAGTAATGGCACAACTACCCTTTCTTGAACTGCAGCCGGAAGAGGATCAAACACCCTTCAAAGTCAGCTTCTTTGTCCGCGATCGCCACGAGACTGTGCTGCGAGAAGTACGGCAACATCTTCGCCGCCATCGCCTGCGGCTGAAGTCAATCTATTCCCATCAGGAGTTTCTTGACATTCTGCCGCTAGCTGCCTCGAAAGGGGATGCGATTCGCCACCTCTCACTCCGCTGGCGGATTCCTCTTGAGAACATTTTGGTGGCAGGCGATTCTGGTAACGATGAGGAAATGCTCAAGGGCCATAATCTCGGCGTTGTAGTTGGCAATTACTCACCGGAATTGGAGCCACTGCGCAGCTACGAGCGCGTCTATTTTGCTGAGGGCCACTATGCTAATGGCATTCTGGAAGCCTTAAAACACTATCGCTTTTTTGAGGCGATCGCTTAA

SEQ ID NO: 13 - skipped

(19) United States
(12) Patent Application Publication
(10) Pub. No.: US 2025/0136534 A1
(43) Pub. Date: May 1, 2025

(54) METHOD FOR PRODUCING VINYL CHLORIDE MONOMER FROM CARBON DIOXIDE

(71) Applicant: Oxy Low Carbon Ventures, LLC, Houston, TX (US)

(72) Inventors: Donald H. POWERS, Houston, TX (US); Tahereh KARIMI, Houston, TX (US); Robert L. ZELLER, III, Seabrook, TX (US); John PACE, Cypress, TX (US); Michael A. GARMON, Manvel, TX (US); Truong Huu NGUYEN, Houston, TX (US)

(21) Appl. No.: 18/692,116

(22) PCT Filed: Sep. 16, 2022
(86) PCT No.: PCT/US2022/076568

§ 371 Date: Mar. 14, 2024

Related U.S. Application Data
(60) Provisional application No. 63/244,864, filed on Sep. 16, 2021.

(51) Int. Cl. C07C 17/389; C12M 1/00

(52) U.S. Cl. CPC C07C 17/389; C12M 21/02; C12M 31/00; C12M 43/06

(57) ABSTRACT

A method for producing a purified stream of vinyl chloride monomer, the method comprising (i) providing a gaseous stream that includes vinyl chloride monomer, carbon dioxide, and water; (ii) dehydrating the gaseous stream to remove substantially all of the water from the stream to thereby produce a substantially dehydrated stream; (iii) liquifying at least the vinyl chloride monomer within the substantially dehydrated stream; and (iv) separating the carbon dioxide as a gaseous stream from the liquid vinyl chloride monomer to thereby produce a purified vinyl chloride monomer stream.

This application claims the benefit of U.S. provisional application Ser. No. 63/244,864 filed on Sep. 16, 2021, which is incorporated herein by reference.

### FIELD OF THE INVENTION

Embodiments of the present invention provide a method and system for converting carbon dioxide to vinyl chloride monomer. Other embodiments are directed toward methods for separating carbon dioxide from vinyl chloride monomer.

### BACKGROUND OF THE INVENTION

Vinyl chloride, which is often referred to as vinyl chloride monomer (VCM), is one of the largest chemical feed stocks produced throughout the world. Polymerization of vinyl chloride produces poly(vinylchloride) (PVC), which is one of the top produced synthetic polymers in the world.

VCM is commonly produced by thermally cracking ethylene dichloride (EDC), which itself is typically produced by halogenating ethylene. This thermal cracking process is often operated in conjunction with an oxychlorination process, which reacts hydrogen chloride obtained as a by-product from the thermal cracking process with ethylene and oxygen to produce additional EDC.

In view of the demand for VCM and the possible scarcity of ethylene, there is a need for alternative routes for the production of VCM.

### SUMMARY OF THE INVENTION

One or more embodiments of the present invention provide a method for producing a purified stream of vinyl chloride monomer, the method comprising (i) providing a gaseous stream that includes vinyl chloride monomer, carbon dioxide, and water; (ii) dehydrating the gaseous stream to remove substantially all of the water from the stream to thereby produce a substantially dehydrated stream; (iii) liquifying at least the vinyl chloride monomer within the substantially dehydrated stream; and (iv) separating the carbon dioxide as a gaseous stream from the liquid vinyl chloride monomer to thereby produce a purified vinyl chloride monomer stream.

Other embodiments of the present invention provide a process comprising (i) providing a gaseous stream including greater than 1% by volume carbon dioxide; (ii) providing water; (iii) converting the carbon dioxide and the water to an organic intermediate and oxygen gas in the presence of light; (iv) separating the oxygen gas from the organic intermediate; (v) converting the organic intermediate to vinyl chloride and carbon dioxide after said step of separating the oxygen gas from the organic intermediate; and (vi) separating the vinyl chloride from the carbon dioxide.

Yet other embodiments of the present invention provide a system for producing vinyl chloride, the system comprising (i) a first bioreactor including photosynthetic microorganisms that convert carbon dioxide to an organic intermediate, said first bioreactor having a carbon dioxide inlet and an outlet for the organic intermediate; and (ii) a second bioreactor in fluid communication with the first bioreactor and including microorganisms that convert the organic intermediate produced in the first bioreactor to vinyl chloride, said second bioreactor having an outlet for gaseous materials including vinyl chloride, and said second bioreactor having an outlet for fluid materials including unreacted organic intermediate.

### BRIEF DESCRIPTION OF THE DRAWINGS

FIG. 1 is a schematic view of a system for practicing embodiments of the invention.

FIG. 2 is a schematic view of a subsystem for delivering carbon dioxide within embodiments of the present invention.

FIG. 3 is a schematic view of an alternate system including a second photosynthetic bioreactor for practicing embodiments of the invention.

FIG. 4 is a schematic view of a system for including a oxygen-fuel combustion system for practicing embodiments of the invention.

FIG. 5 is a schematic view a system including upstream carbon dioxide purification for practicing embodiments of the invention.

### DETAILED DESCRIPTION OF ILLUSTRATIVE EMBODIMENTS

Embodiments of the invention are based, at least in part, on the discovery of a method for biosynthesizing vinyl chloride from carbon dioxide at industrially significant levels. According to the embodiments of the invention, carbon dioxide is first photosynthetically converted to an organic intermediate with the by-product production of oxygen gas. The by-product oxygen gas can be separated from the organic intermediate, and the organic intermediate is then biologically converted to vinyl chloride within a product stream that includes carbon dioxide, water, and hydrogen gas. According to embodiments of the invention, the product stream is dehydrated, and then the carbon dioxide is separated from the vinyl chloride by cryogenically distilling the carbon dioxide. The carbon dioxide is then advantageously routed back to upstream processes to thereby provide a process having high carbon efficiency. Sub-embodiments of the invention include methods for separating VCM from streams that include VCM, carbon dioxide, and optionally other constituents such as, but not limited to, other chlorinated organics including those chlorinated organics that are by-products of the biosynthesis of VCM.

### Process and System Overview

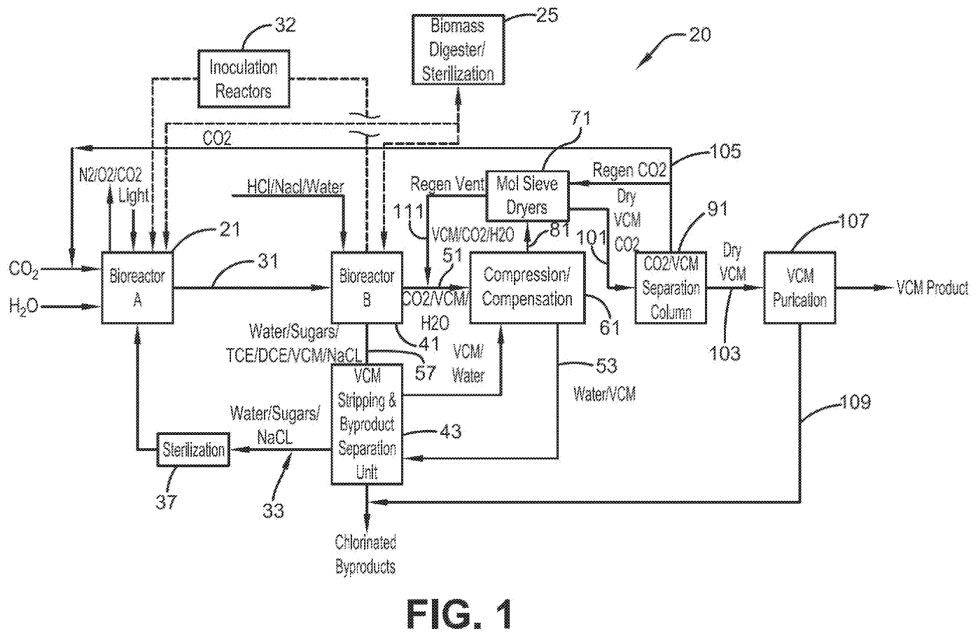

Embodiments of the invention can be described with reference to FIG. 1, which depicts a system 20 for converting carbon dioxide to vinyl chloride. The system includes a first bioreactor 21 followed in series by a second bioreactor 41. First bioreactor 21 is in fluid communication, either directly or indirectly, with second bioreactor 41 via intermediate-product conduit 31. Second bioreactor 41 is also in fluid communication with first bioreactor 21 via intermediate-recycle conduit 33. A compression unit 61 is downstream of second bioreactor 41 and is in fluid communication, either directly or indirectly, with second bioreactor 41 via conduit 51. A molecular sieve unit 71 is downstream of compression unit 61 and is in fluid communication, either directly or indirectly, with compression unit 61 via conduit 81. A cryogenic separation unit 91 is downstream of molecular sieve unit 71 and is in fluid communication, either directly or indirectly, with dehydration station 91 via conduit 101.

According to embodiments of the invention, first bioreactor 21 includes a photosynthetic organism culture (i.e. photosynthetic microorganisms) that converts carbon dioxide and water fed to bioreactor 21 to an organic intermediate. This conversion takes place in the presence of light energy that is supplied to first bioreactor 21. The synthesis of the organic intermediate takes place in the presence of excess water, which acts as reaction medium, and the excess water acts as the carrier for the intermediate product stream. In one or more embodiments, the organic intermediate is soluble in the water. As the skilled person will appreciate, the photosynthetic organism culture can be supplied to bioreactor 21 from an inoculation reactor 23.

Oxygen gas is produced as a by-product during formation of the organic intermediate within bioreactor 21. The oxygen gas can be separated from the organic intermediate prior introducing the intermediate product stream to second bioreactor 41. For example, the oxygen gas can be vented out of first bioreactor 21 together with other volatiles within the reactor such as nitrogen gas.

The organic intermediate is transferred from first reactor 21, either directly or indirectly, within an intermediate-product stream to second bioreactor 41 via intermediate-product conduit 31. In one or more embodiments, the intermediate-product stream can be filtered as the stream exits bioreactor 21. During operation, filtering of the intermediate-product stream as the stream leaves bioreactor 21 can prevent transfer of any media that is used to immobilize the photosynthetic microorganisms and thereby help prevent transfer of the microorganisms from first bioreactor 21 to second bioreactor 41.

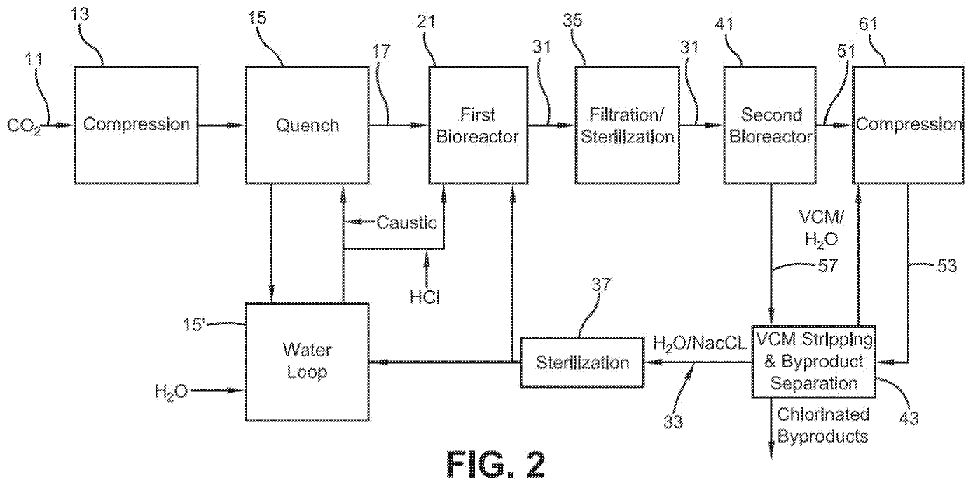

In addition to or in lieu of filtering the intermediate product stream as the stream leaves bioreactor 21, the intermediate-product stream can be filtered and/or sterilized at one or more intermediate units positioned between bioreactor 21 and bioreactor 41. For example, and with reference to FIG. 2, optional sterilization unit 35 can be positioned between first bioreactor 21 and second bioreactor 41. Unit 35 may include a filtration unit. In addition to or in lieu of a filtration unit, unit 35 may include a centrifugation unit. Or, in other embodiments, unit 35 may include, in addition to or in lieu of filtration or centrifugation, a clarification unit (e.g. a settling tank). In lieu of or in addition to filtration, centrifugation, and/or clarification, unit 35 may include a sterilization unit. For example, the sterilization unit may take advantage of UV sterilization, heat, or gamma radiation to treat the intermediate-product stream, which may be done in order to prevent the introduction of any live microorganisms from first bioreactor 21 into second bioreactor 41.

According to embodiments of the invention, second bioreactor 41 includes a vinyl chloride-producing organism culture (i.e. vinyl chloride-producing organisms) that converts the organic intermediate and hydrogen chloride, which is to fed bioreactor 41, to vinyl chloride.

As the skilled person appreciates, several sub-systems can be designed for introducing the microorganism cultures to the respective bioreactors. Those skilled in the art can readily design appropriate systems for accomplishing these goals. For example, and with reference to Figures, the appropriate microorganisms can be supplied to bioreactor 21 and/or bioreactor 41 from an inoculation unit 23, which may also be referred to as inoculation reactor 23. Inoculation unit 23 may include separate chambers or vessels for the respective microorganisms, or separate units may be provided for the respective microorganisms. Likewise, it may be desirable to remove biomass from one or more of the bioreactors. In one or more embodiments, the systems of the present invention may include a biomass digestion unit 25 where biomass obtained from either or both of the bioreactors can be removed from any immobilization support media and removed from the system. During operation, bio-mass digestion unit 25 can be in fluid communication with either or both of bioreactors 21, 41, or the bio-mass (optionally together with the immobilization materials) can be manually removed from the respective reactors. In one or more embodiments, the biomass can be converted to nutrients, such as amino acids, and returned to the bioreactors as a source of nutrient for the microorganisms. Alternatively, the biomass can be removed from the system and directed toward other uses such as fertilizers and the like.

Carbon dioxide is produced as a by-product of vinyl chloride synthesis within bioreactor 41, and the vinyl chloride and carbon dioxide, as well as optional hydrogen gas, are removed from second bioreactor 41 as a gaseous product stream together with contaminant water vapor via conduit 51. A liquid effluent stream also exits second bioreactor 41 via conduit 57. This liquid effluent stream, which can include water and unreacted organic intermediates, can be routed back to first bioreactor 21 via organic intermediate-recycle conduit 33.

The liquid effluent stream may also include chlorinated organic compounds including vinyl chloride and chlorinated byproducts of the reaction within second reactor 41. It is therefore desirable to remove any chlorinated organic compounds before recycling the water and organic intermediates back to first bioreactor 21. Also, this liquid effluent stream may also include inorganic salts such as sodium chloride. It may also be desirable to separate these inorganic compounds from the stream prior to recycling the water and intermediates back to first bioreactor 21. Separation of the chlorinated organic compounds, as well as the inorganic salts, can take place at VCM stripping and by-product separation unit 43. Also, recycle stream may undergo filtration and/or sterilization at unit 37 prior to delivery to first bioreactor 21 in order to address or eliminate any microorganism cross-contamination between the reactors. This filtration and/or sterilization may take advantage of the same types of technologies as unit 35 and therefore the discussion above with respect to unit 35 is incorporated herein. As the skilled person appreciates, it may be useful to ensure that vinyl chloride-producing microorganisms do not migrate into first bioreactor 21.

As noted above, the gaseous product stream exiting second bioreactor 41 is routed downstream of second bioreactor 41 to compression unit 61, either directly or indirectly, via conduit 51. Within compression unit 61, the gaseous product stream is pressurized to facilitate transfer of the gaseous product stream to and through downstream processes. In one or more embodiments, pressurization of the gaseous product stream may be followed by cooling of the pressurized stream, which results in condensing at least a portion of the water contained within the gaseous product stream. The condensed water is removed from the product stream and optionally routed back to bioreactor 21 via conduit 53.

The pressurized gaseous product stream exiting compression unit 61 is routed downstream to molecular sieve unit 71 where water is removed from the gaseous product stream to levels that will not have a deleterious impact on downstream separations that take place within cryogenic separation unit 91.

The dehydrated stream then undergoes cryogenic separation within separation unit 91 to provide a concentrated vinyl chloride stream, which may also be referred to as a vinyl chloride-rich stream, which may be carried by conduit 103 to downstream polishing at polishing unit 107. For example, this vinyl chloride-rich stream can be routed to a downstream distillation column where vinyl chloride is separated from heavy constituents that may be present in the vinyl chloride-rich stream such as trichloroethylene, dichloroethylene, and perchloroethylene.

Cryogenic separator 91 also produces a concentrated carbon dioxide stream, which may also be referred to as a purified carbon dioxide stream, and this concentrated carbon dioxide stream can be routed to upstream units via conduit 105. For example, the concentrated carbon dioxide stream can be routed back to first bioreactor 21 for conversion to organic intermediates. Also, concentrated carbon dioxide stream can be routed to molecular sieve unit 71 where it can be employed to regenerate the molecular sieves as will be described in greater detail below.

### Intermediate Photosynthetic Bioreactor

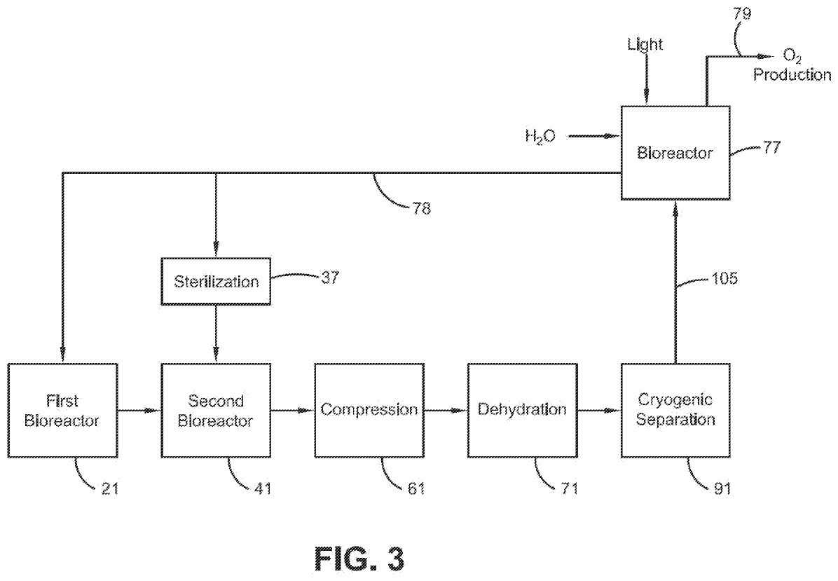

In alternative embodiments, which may be described with reference to FIG. 3, the purified carbon dioxide stream produced by cryogenic separator 91 can be routed, via conduit 105, to an intermediary bioreactor 77 (which may be referred to as second photosynthetic bioreactor 77) that includes a photosynthetic organism culture that photosynthetically converts the carbon dioxide to an organic intermediate and oxygen gas. Advantageously, since the carbon dioxide feed stream to bioreactor 77 is a purified carbon dioxide stream (via cryogenic separator 91), the gaseous by-product stream leaving second photosynthetic bioreactor 77 includes a relatively pure oxygen gas stream, which can be routed via conduit 79. The skilled person will appreciate, within the context of this invention, that relatively pure oxygen streams include those streams that are substantially devoid of nitrogen gas and argon gas, which would otherwise require complicated and potentially expensive processes to separate (e.g. air separation techniques) the nitrogen and argon from the oxygen gas. The presence of carbon dioxide, however, within the relatively pure oxygen gas streams defined herein is not deleterious and therefore may be present within the relatively pure oxygen steams, unless otherwise stated, since carbon dioxide can be more readily separated from the oxygen gas streams. Consistent with first bioreactor 21, intermediary bioreactor 77 produces an effluent stream, which may include organic intermediate and water, that can be routed back, via conduit 78, to first bioreactor 21 and/or second bioreactor 41. As generally shown, this effluent stream may undergo filtration and/or sterilization at unit 37 as described with reference to FIG. 2.

### Carbon Dioxide Pretreatment

With reference now to FIG. 2, the processes of the present invention may include treating the carbon dioxide input stream (i.e. conditioning of the stream prior to providing the stream to bioreactor 21). In one or more embodiments, the carbon dioxide input stream, which is carried by conduit 11, may be pressurized at compressor 13. In one or more embodiments, pressurization of the carbon dioxide input stream (e.g. within compressor 13) achieves sufficient pressure to overcome counter forces within first bioreactor 21 so that inert gases (e.g. nitrogen) within the input stream can ultimately enter the head space of the reactor. In one or more embodiments, the carbon dioxide input stream is pressurized to a pressure of from about 2 to about 20 psig, in other embodiments from about 3 to about 18 psig, and in other embodiments from about 5 to about 15 psig.

Also, as best shown in FIG. 2, the carbon dioxide input stream can be cooled at quencher 15 prior to delivery to bioreactor 21 via conduit 17. As the skilled person will appreciate, quencher 15 may include a water-cooled unit that includes a quench water loop 15′, which may include one or more heat exchangers for cooling the water. In one or more embodiments, the carbon dioxide input stream is cooled to a temperature below that which would otherwise have a deleterious impact on the microorganism culture within bioreactor 21. In one or more embodiments, carbon dioxide input stream is cooled to a temperature of from about 10 to about 80° C., in other embodiments from about 20 to about 60° C., and in other embodiments from about 30 to about 50° C. prior to delivery to bioreactor 21.

Inasmuch as the carbon dioxide input streams of one or more embodiments may include appreciable amounts of water, and at least a portion of the water will be condensed via the cooling cycle at quencher 15, water from quencher 15 can be fed to first bioreactor 21, which consumes substantial amounts of water. In one or more embodiments, the water employed at quencher 15 and/or the water stream routed to first bioreactor 21 can be treated with caustic to adjust the pH of the water. In one or more embodiments, the water employed at quencher 15 and/or water routed to first bioreactor 21 from quencher 15 is adjusted to a pH of greater than 5.5, in other embodiments greater than 6.0, and in other embodiments greater than 6.5 (e.g. in the range 5.5-8.0 or 6.0-7.5). It is anticipated that caustic treatment of the water will form carbonates, such as sodium carbonate, which not only benefits first bioreactor 21 relative to pH control, but also offers a source of additional carbon dioxide in the form of sodium carbonate and/or sodium bicarbonate. Those skilled in the art can readily adjust the conditions and/or provide additional ingredients (e.g. hydrochloric acid) to yield a desirable balance between sodium carbonate and sodium bicarbonate. In particular embodiments, the caustic soda provided to the quench water within quencher 15 derives from other processes that can be integrated with practice of the present invention.

In yet other embodiments, the liquid effluent stream exiting second bioreactor 41, which as described relative to other embodiments can be routed back to first bioreactor 21, can optionally be routed to quencher 15. In one or more embodiments, liquid effluent stream exiting second bioreactor 41 is first treated at sterilization station 37 prior to routing to quencher 15.

### Carbon Dioxide Feed Stream to First Bioreactor

The process of the present invention can advantageously convert carbon dioxide from a variety of gaseous sources, which may be referred to as carbon dioxide input streams, to useful intermediates that can be converted to vinyl chloride. In one or more embodiments, the carbon dioxide is provided to the system by a carbon dioxide input stream that includes greater than 1% vol, in other embodiments greater than 3% vol, in other embodiments greater than 5% vol, and in other embodiments greater than 10% vol carbon dioxide. In one or more embodiments, the carbon dioxide input stream is or derives from an exhaust stream of a combustion process (i.e. a flue gas stream). As the skilled person appreciates, the composition of the exhaust stream can vary based upon several factors including the design of the combustion process and fuel being burned in the combustion process. For example, the flue gas stream can derive from coal-fired furnaces, gas-fired furnaces, turbine-powered generators, and oxy-fuel combustion processes.

In one or more embodiments, the carbon dioxide input stream can derive from an exhaust stream of an oxy-fuel combustion process, which may also be referred to as oxycombustion. Those skilled in the art appreciate that these processes include the combustion of fuels (e.g. hydrocarbons) in the substantial absence of nitrogen and argon. For example, these processes may include combustion processes where substantially pure oxygen (i.e. substantially free of nitrogen and argon gas), or a mixture of pure oxygen and recycled flue gas, is fed to the combustion process. As a result, the combustion products are mostly carbon dioxide and water and substantially low in nitrogen by-products or argon. Advantageously, because the carbon dioxide input stream from an oxycombustion process includes significant levels of carbon dioxide, and is substantially devoid of nitrogen and oxygen, the gaseous by-product stream from photosynthetic bioreactor would include substantially high concentrations of oxygen gas together with any unreacted carbon dioxide. The gaseous by-product stream from photosynthetic bioreactor can then be recycled back to the oxycombustion unit as fuel within the oxycombustion process, with any unreacted carbon dioxide providing cooling to the oxycombustion process.

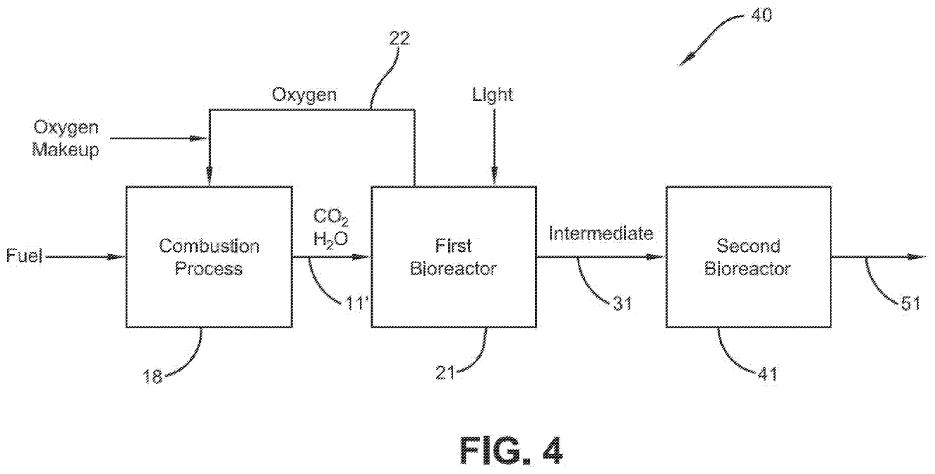

For example, and with reference to FIG. 4, an embodiment of the invention includes a carbon dioxide input stream from an oxycombustion unit 18. As with the previous embodiments, carbon dioxide is photosynthetically converted to an organic intermediate with by-product oxygen gas with first bioreactor 21. The by-product oxygen gas stream, which includes oxygen gas and unreacted carbon dioxide, is routed, via conduit 22, to oxycombustion unit 18. Also, although not shown, the carbon dioxide input stream from oxycombustion unit 18 can be cooled and pressurized as described above with respect to the other embodiments (see e.g. FIG. 2). The intermediate products produced in first bioreactor 21 are routed downstream to second bioreactor 41 in a manner consistent with other embodiments.

In yet other embodiments, relatively pure carbon dioxide streams can be fed to first bioreactor 21. Relatively pure carbon dioxide streams can be obtained from several sources and generally include those streams that contain greater than 90 vol %, in other embodiments greater than 95 vol %, and in other embodiments greater than 99 vol % carbon dioxide. As explained above, where relatively pure carbon dioxide streams are used, the process of the present invention produces relatively pure oxygen gas streams (i.e. substantially free of nitrogen or argon gas) as a by-product output from first bioreactor 21. These relatively pure oxygen gas streams can be used, for example, in industrial applications such as the oxychlorination of ethylene.

In one or more embodiments, a relatively pure carbon dioxide stream is produced as a step of the present invention and used as an input stream. For example, an input stream containing carbon dioxide can be purified and/or concentrated prior to introducing the stream to first bioreactor 21. In one or more embodiments, the carbon dioxide input stream can be purified by using, for example, amine scrubbing and stripping techniques. As with one or more of the previous embodiments, by providing a purified carbon dioxide stream to the first bioreactor, relatively high-grade oxygen gas streams can be produced as the by-product stream exiting the first bioreactor. The skilled person will appreciate that a variety of carbon dioxide removal and separation techniques can be used in addition to or in lieu of amine scrubbing and stripping techniques to purify and/or concentrate a carbon dioxide stream. These techniques include, but are not limited to, membrane separation, solid sorbents, and the use of other solvent chemistries such as potassium carbonate.

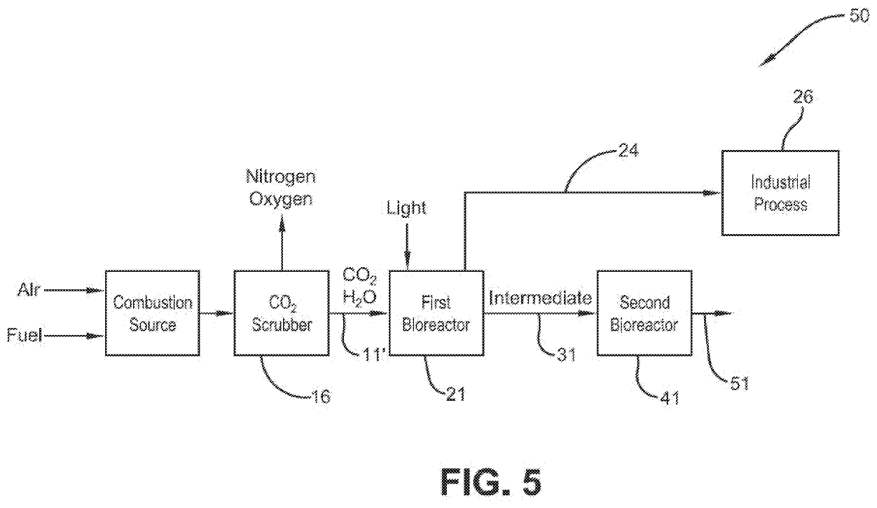

An exemplary embodiment can be described with reference to FIG. 5, which shows a system 50 including first bioreactor 21 that receives a carbon dioxide input stream from a carbon dioxide purification unit 16. Purification unit 16 produces a purified carbon dioxide stream that is introduced to first bioreactor 21 via conduit 11′. Although not shown, the carbon dioxide input stream from the combustion unit can be cooled and pressurized as described above with respect to the other embodiments (see e.g. FIG. 2). The carbon dioxide is photosynthetically converted to an organic intermediate with by-product oxygen gas in bioreactor 21. The by-product oxygen gas is routed, either directly or indirectly, via conduit 24, to, for example, an industrial process 26 requiring relatively high purity oxygen. In one or more embodiments, vinyl chloride produced at bioreactor 41 can also be routed to industrial process 26. Although not shown in FIG. 5, it will be appreciated that the vinyl chloride streams from second bioreactor 41 undergo downstream processing as described with regard to other embodiments including, but not limited to, compression, dehydration, and cryogenic separation.

Again, in those embodiments where the photosynthetic bioreactor yields a relatively pure oxygen by-product stream, which can derive from the use of relatively pure carbon dioxide streams, the relatively pure oxygen streams can be used in industrial applications. In one or more embodiments, the gaseous stream exiting the photosynthetic bioreactor can undergo carbon dioxide removal to remove any unreacted carbon dioxide in the oxygen gas stream. Then, the oxygen gas stream can be routed to the desired industrial applications. For example, the oxygen gas stream from these embodiments can be routed to an oxychlorination unit, where ethylene is reacted with hydrochloric acid in the presence oxygen gas.

### Compression of Vinyl Chloride Product Stream

As suggested above, the vinyl chloride product stream is pressurized at compression unit 61, which may be referred to as a pressurization unit 61. Conventional technology and techniques can be employed to pressurize this gaseous stream including, but not limited to, multi-stage compression with inter-stage cooling. In one or more embodiments, pressurization within compression unit 61 includes greater than one, in other embodiments greater than two, and in other embodiments greater than three stages of compression to achieve the desired pressurization of the product stream.

In one or more embodiments, the vinyl chloride product stream is pressurized within unit 61 to a pressure of greater than 75 psig, in other embodiments greater than 125 psig, and in other embodiments greater than 150 psig. In these or other embodiments, the vinyl chloride product stream is pressurized to a pressure of from about 75 to about 225 psig, in other embodiments from about 125 to about 210 psig, and in other embodiments from about 150 to about 200 psig.

In conjunction with pressurization, which increases the temperature of the vinyl chloride product stream, the pressurized product stream can be cooled. For example, where multi-stage pressurization techniques are employed, the stream can be cooled between pressurization stages using conventional techniques such as, but not limited to, the use of local water within a heat exchanger. In one or more embodiments, cooling water having a temperature of from about 60 to about 120° F., in other embodiments from about 70 to about 110° F., and in other embodiments from about 80 to about 110° F. can be employed as a counter stream within a heat exchanger. Advantageously, transfer of heat away from the product stream results in condensation of at least some of the water from the vinyl chloride stream. Those skilled in the art appreciate that condensation of water within the gaseous product stream occurs at higher pressures and lower temperatures, and that temperature and pressure are intertwined in achieving the desired result. In particular embodiments, the pressurized stream is cooled using a refrigerated stream, such as a propylene refrigeration or chilled water stream (e.g. heat is exchanged with a refrigerated counter stream within a heat exchanger). In particular embodiments, this refrigerated stream may have a temperature of less than 60° F., in other embodiments less than 50° F., and in other embodiments less than 40° F. In particular embodiments, the step of cooling against a refrigerated stream takes place after the final stage of pressurization within pressurization unit 61. In one or more embodiments, this step of cooling provides a pressurized product stream having a temperature of from about 35 to about 70° F., in other embodiments from about 40 to about 65° F., and in other embodiments from about 45 to about 60° F.

### Drying of Vinyl Chloride Product Stream

In one or more embodiments, molecular sieve unit 71, which may also be referred to as drying unit 71, may include one or more water adsorption beds that include drying material such as, for example, molecular sieves. As those skilled in the art will appreciate, multiple beds may be used so that the adsorption process can be continuous while one or more beds undergo desorption or regeneration. For example, in a two-bed system, one bed may be operated while the other bed is being regenerated.

In one or more embodiments, the molecular sieves have a pore opening of less than 5 Å, in other embodiments less than 4 Å, and in other embodiments less than 3. In particular embodiments, a 3A molecular sieve is employed.

In one or more embodiments, molecular sieve beds are pressure swing adsorption system. In other embodiments, the molecular sieve beds are a temperature swing adsorption system.

In one or more embodiments, the vinyl chloride product stream undergoes a pressure drop of from about 0.5 to about 15 psig, in other embodiments from about 1 to about 12 psig, and in other embodiments from about 3 to about 10 psig during treatment within molecular sieve unit 71.

In one or more embodiments, the vinyl chloride product stream, after treatment within drying unit 71, is characterized by a water content of less than 20 ppm (vol), in other embodiments less than 10 ppm (vol), and in other embodiments less than 5 ppm (vol).

### Cryogenic Carbon Dioxide Separation

In one or more embodiments, separation at unit 91 takes place by cryogenic separation, which may also be referred to as cryogenic distillation or cryogenic fractionation. According to these systems and techniques, the dehydrated vinyl chloride product stream is at least partially liquefied by cooling, optionally in combination with changes in pressure, to form a stream that includes liquified vinyl chloride and optionally liquefied carbon dioxide. This stream, which may be a mixed phase stream, can then be provided to a distillation column where the carbon dioxide is separated as an overhead stream. In one or more embodiments, at least a portion of the overhead carbon dioxide stream can be condensed and returned to the column as reflux. The purified carbon dioxide stream is routed from separation unit 91 via conduit 105. The vinyl chloride, which remains after separation of the carbon dioxide, is removed from separation unit 91 as a liquid stream via conduit 103.

In one or more embodiments, the dehydrated vinyl chloride stream undergoes separation within unit 91 at a pressure of greater than 75, in other embodiments greater than 100, and in other embodiments greater than 150 psig. In these or other embodiments, the dehydrated vinyl chloride stream undergoes separation within unit 91 at a pressure of from about 75 to about 225, in other embodiments from about 100 to about 215, and in other embodiments from about 150 to about 200 psig. In conjunction therewith, the column is operated so that the overhead streams have a temperature of less than −30° F., in other embodiments less than −35° F., and in other embodiments less than −40° F. In these or other embodiments, the column is operated to achieve overhead temperatures of from about −100 to about −30° F., in other embodiments from about −70 to about −35° F., and in other embodiments from about −60 to about −40° F. In these or other embodiments, the bottoms, which include the target vinyl chloride, exit the column at temperatures of greater than 90° C., in other embodiments greater than 120° C., and in other embodiments greater than 140° C.

Molecular Sieve Regeneration with Carbon Dioxide

As indicated above, a purified carbon dioxide stream is routed from separation unit 91 via conduit 105. As shown in FIG. 1, this stream can be routed to back to first bioreactor 21. In addition, at least a portion of the purified carbon dioxide stream can be used to regenerate the molecular sieve beds employed within drying unit 71. The regenerated vent stream exiting the molecular sieve bed being regenerated can advantageously be recycled back to the system for recovery of any residual vinyl chloride that may be captured within the drying beds. For example, in one or more embodiments, the regenerated vent stream, which is carried by conduit 111, can be routed back to compression unit 61where the stream will undergo compression, and then the pressurized stream can be sent to downstream drying in drying unit 71, and then to carbon dioxide separation in unit 91. Those skilled in the art will appreciate that regenerated vent stream exiting the regeneration of molecular sieve unit 71 will carry appreciable water, which could have a deleterious impact on the overall efficiency of drying units 71 when the regenerated vent stream is recycled back to drying unit 71. In order to alleviate these issues, appreciable water is removed from the stream within pressurization unit 61. As indicated above, pressurization and cooling within unit 61 condenses water contained within the streams being treated to thereby allow for removal of the water from the streams. At least for purposes of facilitating recycle of the regenerated vent streams, greater than 50% (vol), in other embodiments greater than 60% (vol), and in other embodiments greater than 70% (vol) of the water within the stream entering pressurization unit 61 is condensed from the stream prior to the stream being provided to drying unit 71. Those skilled in the art will be able to readily manipulate the techniques and conditions within compression unit 61 to achieve the desired water levels.

### VCM Stripping and Byproduct Separation

As mentioned above, the liquid effluent stream from second bioreactor 41, which can be carried by conduit 57, can include a variety of constituents such as chlorinated organics, inorganic salts, as well as unreacted organic intermediates. Separation of these various constituents into useful recycle streams and/or waste streams can be performed by employing one or more separation processes or steps. With reference to FIG. 1, at least one of these steps takes place at VCM stripping and by-product separation unit 43, which may simply be referred to as separation unit 43. This may include, for example, steam stripping to separate and isolate vinyl chloride and/or other chlorinated byproducts. The effluent from separation unit 43 can then undergo filtration and/or sterilization at filtration/sterilization unit 37. In one or more embodiments, the steam stripping or distillations may take place at temperatures of less than 300° F., in other embodiments less than 250° F., and in other embodiments less than 200° F. Those skilled in the art will be able to readily adjust pressure (i.e. vacuum) to achieve the desired separations.

### Vinyl Chloride Purification

As described above, once carbon dioxide is separated from the vinyl chloride (e.g. within separator 91), the vinyl chloride stream is carried by conduit 103 to downstream optional purification processes at polishing unit 107, which may also be referred to as VCM distillation column 107. In one or more embodiments, the vinyl chloride product stream can be purified by employing conventional distillation techniques that can, for example, separate the target vinyl chloride as an overhead stream from other constituents within the product stream (such as heavier chlorinated byproducts such as, but not limited to, trichloroethylene, dichloroethylene and perchloroethylene), which will remain in the distillation bottoms. These heavier compounds can be routed via conduit 109 to a waste stream.

In one or more embodiments, distillation of the vinyl chloride stream within polishing unit 107 takes place at temperatures of from about 120 to about 170° F., in other embodiments from about 130 to about 165° F., and in other embodiments from about 140 to about 160° F. In conjunction therewith, purification of the vinyl chloride product stream within unit 107 can take place a pressures of from about 75 to about 225 psig, in other embodiments from about 100 to about 215 psig, and in other embodiments from about 150 to about 200 psig.

### Photosynthetic Microorganisms

As set forth above, the first bioreactor contains a photosynthetic organism culture containing one or more types of photosynthetic microorganisms that converts the carbon dioxide and water into an organic intermediate in the presence of light energy. In various embodiments, the photosynthetic microorganisms may be naturally occurring. In other embodiments, the photosynthetic microorganisms may be genetically modified for improved production of a desired organic intermediate. In one or more embodiments, the photosynthetic microorganisms utilized with the first bioreactor may include photosynthetic bacteria such as cyanobacteria. As those skilled in the art appreciate, photosynthetic bacteria consume carbon dioxide and water in the presence of light to fix carbon. Advantageously, the main products of the metabolic pathway of cyanobacteria during aerobic conditions are oxygen and organic intermediates, such as sugars. One of ordinary skill in the art will be able to select a suitable photosynthetic microorganism, without undue experimentation, to produce a desired organic intermediate.

In one or more embodiments, the desired organic intermediate includes sucrose, dextrose, xylose, glucose, fructose, alpha-ketoglutarate, or a mixture thereof.

Exemplary photosynthetic microorganisms include, without limitation, cyanobacteria, algae, and purple bacteria. Useful types of cyanobacteria include photosynthetic prokaryotes that carry out oxygenic photosynthesis. Cyanobacteria useful for the purposes outlined herein are generally well known in the art. (See, e.g., Donald Bryant, The Molecular Biology of Cyanobacteria, published by Kluwer Academic Publishers (1994), the disclosure of which in incorporated herein by reference in its entirety). Representative examples include cyanobacteria in the genus Synechococcus such as Synechococcus lividus and Synechococcus elongatus; and cyanobacteria in the genus Synechocystis such as Synechocystis minervae and Synchocystis Sp PCC 6803. In this regard, U.S. Pat. No. 7,807,427 is incorporated herein by reference in its entirety. Examples of synthetic microorganisms that can be used include those disclosed in U.S. Pat. No. 10,196,627, which is incorporated herein by reference in its entirety. Still other examples include those microorganisms disclosed in U.S. Pat. Nos. 9,914,947 and 9,309,541, which are incorporated herein by reference in their entirety.

In one or more embodiments, the cyanobacteria are genetically modified to express one or more foreign genes encoding one or more enzymes that provide for enhanced production of target organic intermediates. In one or more embodiments, the target organic intermediates include sucrose, dextrose, xylose, glucose, fructose, and alpha-ketoglutarate. As will be apparent to those of skill in the art, the particular genes added to the genome of the cyanobacteria (and enzymes produced) will depend upon the particular target organic intermediate.

In one or more embodiments, the modified photosynthetic microorganism includes a modified nucleotide sequence that produces an enzyme that forms alpha-ketoglutarate from carbon dioxide. In certain embodiments, this modified photosynthetic microorganism expresses an alpha-ketoglutarate permease protein (AKGP) by expressing a non-native AKGP forming nucleotide sequence. In one or more embodiments, the modified microorganism produces a greater amount of the enzyme than produced by a control microorganism lacking the modified nucleotide sequence. This amount may be greater than 1%, in other embodiments greater than 50%, and in other embodiments greater than 75% of the amount produced by a control microorganism lacking the modified nucleotide sequence.

In one or more embodiments, alpha-ketoglutarate (aKG) can be produced by oxidative decarboxylation of isocitrate by isocitrate dehydrogenase (ICD), or by oxidative deamination of glutamate by glutamate dehydrogenase (GDH). Target enzymes for cloning and aKG production in Cyanobacteria may include ICD Enzyme: 1.1.1.42, coding sequence of P. Fluorescens ICD (SEQ ID NO: 1, SEQ ID NO: 2), ICD Enzyme: 1.1.1.42, coding sequence of Synechococcus elongatus PCC794 (SEQ ID NO: 3, SEQ ID NO: 4), and GDH Enzyme: 1.4.1.2, coding sequence of P. Fluorescens (SEQ ID NO: 5, SEQ ID NO: 6).

In one or more embodiments, the enzyme for forming alpha-ketoglutarate is selected from isocitrate dehydrogenase (ICD) protein, a glutamate dehydrogenase (GDH) protein, or a combination thereof.

In certain embodiments, the modified photosynthetic microorganism expresses an ICD protein having an amino acid sequence at least 98%, or at least 95%, or at least 90%, or at least 85% identical SEQ ID NO: 1 by expressing a modified ICD protein nucleotide sequence having a nucleotide sequence at least 98%, or at least 95%, or at least 90%, or at least 85% identical to SEQ ID NO: 2. In certain embodiments, the modified microorganism expresses an ICD protein having an amino acid sequence at least 98%, or at least 95%, or at least 90%, or at least 85% identical SEQ ID NO: 3 by expressing a modified ICD protein nucleotide sequence having a nucleotide sequence at least 98%, or at least 95%, or at least 90%, or at least 85% identical to SEQ ID NO: 4. In certain embodiments, the modified microorganism expresses a GDH protein having an amino acid sequence at least 98%, or at least 95%, or at least 90%, or at least 85% identical SEQ ID NO: 5 by expressing a modified GDH protein nucleotide sequence having a nucleotide sequence at least 98%, or at least 95%, or at least 90%, or at least 85% identical to SEQ ID NO: 6.

In one or more embodiments, the organic intermediate is sucrose. In some of these embodiments, for example, the Cyanobacteria (Synechococcus elongatus, Synechocystis) can be engineered to produce sucrose to serve as a substrate for the growth of the vinyl chloride producing microorganisms. Various methods for the engineering of Synechococcus elongatus PCC 7942 to produce sucrose can include activation of one gene (cscB) and deletion of one gene (GlgC).

In one or more of these embodiments, the modified photosynthetic microorganism expresses a sucrose synthase protein. In one or more embodiments, the sucrose synthase protein has an amino acid sequence at least 98%, or at least 95%, or at least 90%, or at least 85% identical to SEQ ID NO: 9 by expressing a modified sucrose synthase protein nucleotide sequence having a nucleotide sequence at least 98%, or at least 95%, or at least 90%, or at least 85% identical to SEQ ID NO: 10. In one or more of these embodiments, the modified microorganism expresses a sucrose phosphate synthase protein. In one or more embodiments, the sucrose phosphate synthase protein has an amino acid sequence at least 98%, or at least 95%, or at least 90%, or at least 85% identical to SEQ ID NO: 11 by expressing a modified sucrose phosphate synthase protein nucleotide sequence having a nucleotide sequence at least 98%, or at least 95%, or at least 90%, or at least 85% identical to SEQ ID NO: 12.

As used herein, sequence identity is the relationship between two or more amino acid (polypeptide or protein) sequences or two or more nucleic acid (polynucleotide) sequences, as determined by comparing the sequences. Sequence identities or similarities are typically compared over the whole length of the respective sequences. The skilled person appreciates that “identity” refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. The skilled person can readily calculate “identity” and “similarity” by various known methods. For example, methods to determine identity and similarity are codified in publicly available computer programs, such as the BestFit, BLASTP (Protein Basic Local Alignment Search Tool), BLASTN (Nucleotide Basic Local Alignment Search Tool), FASTA (Altschul, S. F. et al., J. Mol. Biol. 215:403-410 (1990), publicly available from NCBI and other sources (BLAST.RTM. Manual, Altschul, S., et al., NCBI NLM NIH Bethesda, Md. 20894), and EMBOSS (European Molecular Biology Open Software Suite). Exemplary parameters for amino acid sequences comparison using EMBOSS are gap open 10.0, gap extend 0.5, Blosum matrix. Exemplary parameters for nucleic acid sequences comparison using EMBOSS are gap open 10.0, gap extend 0.5, DNA full matrix (DNA identity matrix). As the skilled person understands, the DNA/protein sequences among different species can be compared to determine the homology of sequences using online data such as Gene bank, KEG, BLAST and Ensemble. The skilled person may also take into account so-called “conservative” amino acid substitutions, which refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Unless otherwise noted, the term “adapted” or “codon adapted” refers to “codon optimization” of polynucleotides as disclosed herein, the sequence of which may be native or non-native, or may be adapted for expression in other microorganisms. Codon optimization adapts the codon usage for an encoded polypeptide towards the codon bias of the organism in which the polypeptide is to be expressed. Codon optimization generally helps to increase the production level of the encoded polypeptide in the host cell.

In certain embodiments, the modified photosynthetic microorganism includes a delta-glgc (Δglgc) mutant microorganism lacking expression of a glucose-1-phosphate adenylyltransferase protein. Similarly, cyanobacterial cells that lack a functional ADP-glucose pyrophosphorylase enzyme are known as described in U.S. Pat. Nos. 9,309,541 and 9,309,541, which are incorporated herein by reference in their entirety.

### Vinyl Chloride-Producing Microorganisms

As described above, the vinyl chloride-producing organisms include those organisms that are genetically modified to produce vinyl chloride by consuming the organic intermediate produced in the photosynthetic bioreactor. In one or more embodiments, multiple, distinct microorganisms are employed to accomplish a multi-step reaction with different microorganisms impacting separate steps. For example, a first microorganism can be employed to consume the organic intermediate and one or more other reactants such as chlorine-containing reactants to thereby produce a first chlorinated intermediate, and then a second microorganism is employed to consume the first chlorinated intermediate to produce vinyl chloride.

### First Bioreactor (Photosynthetic Bioreactor)

With reference again to the Figures, first bioreactor 21 may include a single reaction vessel or it may include a plurality (i.e. two or more) reaction vessels that may operate in a complementary fashion. For example, the two or more reactor vessels may operate in parallel or in series to facilitate the desired photosynthetic reaction.

The skilled person generally appreciates the appropriate conditions that should be maintained with first bioreactor 21 to sustain the microorganisms and promote the desired photosynthetic reaction. In one or more embodiments, water is both a reactant and serves as the reaction medium within first bioreactor 21.

In one or more embodiments, the reactor medium within the photosynthetic bioreactor is maintained at a temperature of from about 25 to about 70° C., in other embodiments from about 35 to about 60° C., and in other embodiments from about 40 to about 50° C. In these or other embodiments, the reaction medium within the photosynthetic bioreactor is maintained at a pH of from about 5.0 to about 8.5, in other embodiments from about 5.5 to about 8.0, and in other embodiments from about 6.0 to about 7.0.

In one or more embodiments, the photosynthetic bioreactor is substantially devoid of microorganisms that produce or are adapted to produce an efe gene.

In one or more embodiments, the first bioreactor includes at least one inlet for the introduction of at least reactant (e.g. carbon dioxide) into the bioreactor. In these or other embodiments, the first bioreactor includes at least one outlet for removing at least one product or at least one by-product from the bioreactor. In one or more embodiments, first bioreactor 21 includes an outlet for gaseous product/by-product and an outlet for liquid effluent. In one or more embodiments, bioreactor 21 is a closed system but for the inlets and outlets. In other embodiments, bioreactor 21 is an open system. In one or more embodiments, the first bioreactor is selected from a continuous stirred tank reactor, a gas lift reactor, a loop reactor, and fluidized bed reactor. In one or more embodiments, the first bioreactor has a capacity of greater than 10,000 gallons, in other embodiments greater than 100,000 gallons, in other embodiments greater than 1,000,000 gallons.

### Second Bioreactor (Vinyl Chloride-Producing Bioreactor)

With reference again to the Figures, second bioreactor 41 may include a single reaction vessel or it may include a plurality (i.e. two or more) reaction vessels that may operate in a complementary fashion. For example, the two or more reactor vessels may operate in parallel or in series to facilitate the desired reaction of converting the intermediate to vinyl chloride.

The skilled person generally appreciates the appropriate conditions that should be maintained with the second bioreactor to sustain the microorganisms and promote the desired vinyl chloride-forming reaction. In one or more embodiments, water serves as the reaction medium in the second reactor.

In one or more embodiments, the reactor medium within the vinyl chloride-forming bioreactor is maintained at a temperature of from about 25 to about 70° C., in other embodiments from about 35 to about 60° C., and in other embodiments from about 40 to about 50° C. In these or other embodiments, the reaction medium within the vinyl chloride forming bioreactor is maintained at a pH of from about 6.0 to about 9.5, in other embodiments from about 6.5 to about 9.0, and in other embodiments from about 7.0 to about 8.0.

In one or more embodiments, the vinyl chloride forming bioreactor is substantially devoid of microorganisms that produce or are adapted to produce oxygen. For example, the second bioreactor is devoid or substantially devoid of photosynthetic microorganisms (e.g. microorganisms that operate by the Calvin Cycle).

In one or more embodiments, the vinyl chloride-forming bioreactor is maintained under anaerobic conditions. In one or more embodiments, the vinyl chloride-forming bioreactor is maintained in the substantial absence of light energy.

In one or more embodiments, the second bioreactor includes at least one inlet for the introduction of the organic intermediate product stream into the bioreactor. In these or other embodiments, the second bioreactor includes at least one outlet for removing the product (e.g. gaseous outlet for vinyl chloride gas) and at least one by-product from the second bioreactor (e.g. carbon dioxide). In one or more embodiments, the second bioreactor also includes an effluent outlet for the removal of liquid effluent (e.g. water and unreacted organic intermediate). In one or more embodiments, the second bioreactor is selected from a continuous stirred tank reactor, a loop reactor, and fluidized bed reactor. In one or more embodiments, the second bioreactor has a capacity of greater than 10,000 gallons, in other embodiments greater than 100,000 gallons, in other embodiments greater than 1,000,000 gallons. In one or more embodiments, the second bioreactor is adapted to provide a closed system but for the reactant inlet and product or by-product outlet.

In one or more embodiments, the concentration of microorganisms within the second reactor may be quantified based upon the dry cell weight per unit volume of the reactor. For example, in one or more embodiments, the microorganism concentration in the second reactor is greater than 10, in other embodiments greater than 50, and in other embodiments greater than 100 grams dry cell weight per liter.

### Integrated Bioreactor

Alternate embodiments of the present invention employ a single vessel for the bioreactions in lieu of the two bioreactors 21, 41 shown with respect to the other systems described above. This vessel may be referred to as integrated bioreactor. During operation, light energy supplied to the integrated bioreactor is controlled in a manner to produce a light cycle and a dark cycle. During operation, photosynthetic microorganisms within the integrated bioreactor convert carbon dioxide to an organic intermediate during the light cycle, and then during the dark cycle, vinyl chloride-forming microorganisms within the integrated bioreactor convert the organic intermediate to vinyl chloride during the dark cycle. Oxygen gas can be removed from the integrated bioreactor during its production during the light cycle, and vinyl chloride can be removed from the integrated bioreactor during its production during the dark cycle. Consistent with other embodiments, vinyl chloride may be coproduced with carbon dioxide, and the vinyl chloride can be separated from the carbon dioxide in downstream processes as described above (i.e. compression at unit 61, dehydration at unit 71, and cryogenic separation at unit 91).

### Techniques for Forming Recombinant Microorganisms

In certain embodiments, the nucleotide sequence for expressing the intermediate forming enzyme or for the efe is inserted into a microbial expression vector. In various embodiments, the microbial expression vector may include a bacterial vector plasmid, a nucleotide guide of a homologous recombination system, an antibiotic-resistant system, an aid system for protein purification and detection, a CRISPR CAS system, a phage display system, or a combination thereof.

As set forth above, in one or more embodiments, multiple copies of the efe expressing nucleotide sequence may be inserted into the vinyl chloride forming microorganisms. Similarly, multiple copies of the intermediate enzyme expressing nucleotide sequence may be inserted into the photosynthetic microorganisms. The number of copies of a gene inserted into a vector and/or a host genome is referred to herein as the “copy number.” In certain embodiments, the efe expressing nucleotide sequence has a copy number in the microbial expression vector of greater than 1, in other embodiments greater than 10, in other embodiments greater than 100, and in other embodiments greater than 250. As will be apparent, expressing multiple copies of the efe expressing nucleotide sequence can increase vinyl chloride yields, thus reducing the volume and cost of ethylene production on a commercial scale.

In certain embodiments, the microbial expression vector includes at least one microbial expression promoter. As will be understood by those of skill in the art, the microbial expression promotor is a nucleotide sequence that initiates transcription of a later, usually adjacent, sequence of DNA and may be constitutive or inducible. In certain embodiments, the at least one microbial expression promoter may include, without limitation, a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof. In certain embodiments, at least one promoter inducer may be added to the bioreactors or the reaction media within the bioreactors to control the amount of the organic intermediate and/or the amount of the ethylene being produced. In certain embodiments, the promoter inducer includes lactose, xylose, IPTG, cold shock, heat shock, or a combination thereof.

In one or more embodiments, the ICD and GDH genes may be synthesized using gBlocks™ gene fragments cloned into a pSyn6 plasmid construct (pSyn6_ICD and pSyn6_GDH). For cloning into a pSyn6 plasmid, the S. elongatus ICD coding sequence is flanked by an N-terminal HindIII and C-terminal BamHI recognition sites (SEQ ID NO: 4). Using the plasmid constructs, the ICD and GDH genes can be cloned into an unmodified S. elongatus or an S. elongatus Δglgc mutant strain (see Example 2). Between 1-3 copies of the target genes may be transformed. Cloning of the ICD and GCH genes can be confirmed by PCR and sequencing. aKG synthesis and quantification can be evaluated by SDS-PAGE, Western Blot, and Ethylene production assays.

In one or more embodiments, creating glycogen mutant strains of Cyanobacteria will change the bacteria's pathway to produce, and also secrete, higher concentrations of keto acids such as aKG.Glycogen mutant Cyanobacteria can be generated by creating glycogen deficient strains via mutations of the glgc gene (Δglgc). For example, an Ampicillin Resistance (AmpR) gene can be synthesized using gBlocks™ and incorporated into a plasmid construct. The plasmid construct can be transformed into a wild type Cyanobacteria (e.g., Synechocystis, Synechococcus elongatus 2973, Synechococcus elongatus 2434). A portion of the wild type glgc gene can then be replaced by the AmpR gene to create the mutant strains. The Δglgc mutant strains can be confirmed by growth in AmpR containing media, followed by PCR and sequencing.

### Maintaining Level of Volatile Gases

In one or more embodiments, the second reactor includes safe levels of oxygen gas. In particular, the head space in the second reactor and the gaseous outlet stream of the second reactor include safe levels of oxygen gas relative to vinyl chloride. As the skilled person appreciates, commercially acceptable levels of oxygen gas within ethylene streams can be defined by the lower explosion limit (LEL), which takes into account the level of ethylene present. In one or more embodiments, the amount of oxygen within the second reactor (i.e. within the head space of the reactor or in the gaseous outlet stream) is less than the acceptable LEL, in other embodiments less than 80% of the acceptable LEL, and in other embodiments less than 50% of the acceptable LEL.

### Process Characteristics

As described herein, the process of the invention is effective for converting carbon dioxide to vinyl chloride at high carbon efficiency while addressing safety concerns associated with the co-production of vinyl chloride and oxygen by using biosynthetic processes.

In one or more embodiments, the process of the present invention produces vinyl chloride at a production rate of greater than 100, in other embodiments greater than 500, in other embodiments greater than 1000, in other embodiments greater than 1500, in other embodiments greater than 2000, and in other embodiments greater than 2500 μmol/gCDW/hour, where CDW refers to cell dry weight.

Various modifications and alterations that do not depart from the scope and spirit of this invention will become apparent to those skilled in the art. This invention is not to be duly limited to the illustrative embodiments set forth herein.

1. A method for producing a purified stream of vinyl chloride monomer, the method comprising:
(i) providing a gaseous stream that includes vinyl chloride monomer, carbon dioxide, and water;
(ii) dehydrating the gaseous stream to remove substantially all of the water from the stream to thereby produce a substantially dehydrated stream;
(iii) liquifying at least the vinyl chloride monomer within the substantially dehydrated stream; and
(iv) separating the carbon dioxide as a gaseous stream from the liquid vinyl chloride monomer to thereby produce a purified vinyl chloride monomer stream.

2. The method of claim 1, further including pressurizing the gaseous stream that includes vinyl chloride monomer, carbon dioxide, and water to a pressure of greater than 75 psig to thereby form a pressurized stream.

3. The method of claim 2, further including cooling the pressurized stream to thereby condense at least a portion of the water within the pressurized stream.

4. The method of claim 1, where said step of dehydrating the gaseous stream includes drying the stream by passing the stream through molecular sieves.

5. The method of claim 1, where the substantially dehydrated stream includes less than 20 ppm (vol) water).

6. The method of claim 1, where said liquifying at least the vinyl chloride monomer within the substantially dehydrated stream and said separating the carbon dioxide as a gaseous stream takes place by cryogenic separation.

7. The method of claim 6, where said cryogenic separation takes place at a pressure of greater than 75 psig and a temperature of less than −30° F.

8. The method of claim 1, where said separating the carbon dioxide as a gaseous stream from the liquid vinyl chloride monomer produces a purified carbon dioxide stream, and where said purified carbon dioxide stream is introduced to said molecular sieves as a regeneration stream.

9. The method of claim 8, where said regeneration stream is recycled by combining the regeneration stream with the gaseous stream including vinyl chloride monomer, carbon dioxide, and water after introducing the purified carbon dioxide stream to the molecular sieves.

10. (canceled)

11. A process comprising:
(i) providing a gaseous stream including greater than 1% by volume carbon dioxide;
(ii) providing water;
(iii) converting the carbon dioxide and the water to an organic intermediate and oxygen gas in the presence of light;
(iv) separating the oxygen gas from the organic intermediate;
(v) converting the organic intermediate to vinyl chloride and carbon dioxide after said step of separating the oxygen gas from the organic intermediate; and
(vi) separating the vinyl chloride from the carbon dioxide.

12. The process of claim 11, where said step of separating the vinyl chloride from the carbon dioxide includes liquefying the vinyl chloride and separating the carbon dioxide as a gaseous stream from the vinyl chloride.

13. The process of claim 11, where the vinyl chloride and carbon dioxide form a product stream, and further comprising the step of dehydrating the product stream prior to said step of separating the vinyl chloride from the carbon dioxide.

14. The process of claim 11, where said step (v) of converting the organic intermediate to vinyl chloride and carbon dioxide takes place in the presence of excess water from said step (iii) of converting carbon dioxide and water to an organic intermediate, and where said step of (v) converting the organic intermediate to vinyl chloride and carbon dioxide consumes only a portion of the organic intermediate formed in said step (iii) of converting carbon dioxide and water to an organic intermediate and oxygen gas, and further comprising the step of routing the excess water and organic intermediate from said step (v) of converting the organic intermediate to vinyl chloride and carbon dioxide back to said step (iii) of converting the carbon dioxide and water to an organic intermediate.

15. The process claim 11, where said step (iii) of converting carbon dioxide and water to an organic intermediate and oxygen gas takes place within a first bioreactor that contains a microorganism that produces an organic intermediate and oxygen gas from the carbon dioxide in the presence of light energy.

16. (canceled)

17. The process of claim 11, further comprising the step of compressing a flue gas stream from a combustion step to form the stream containing carbon dioxide.

18. (canceled)

19. (canceled)

20. (canceled)

21. The process of claim 11, where said step of (v) converting the organic intermediate to vinyl chloride and carbon dioxide takes place within a second bioreactor that contains one or more microorganisms that produce one or more enzymes for converting the organic intermediate to vinyl chloride and carbon dioxide.

22. The process of claim 11, where said step (v) of converting the organic intermediate to vinyl chloride and carbon dioxide takes place in the substantial absence of light energy.

23. The process of claim 11, where said step of converting carbon dioxide and water to an organic intermediate and oxygen gas takes place within a first bioreactor.

24. (canceled)

25. The process of claim 11, where said step of converting the organic intermediate to vinyl chloride and carbon dioxide after said step of separating the oxygen gas from the organic intermediate takes place within a second bioreactor.

26. The process of claim 11, where said step of separating the vinyl chloride from the carbon dioxide takes place under cryogenic conditions.

27. The process of claim 11, where said step of separating the vinyl chloride from the carbon dioxide produces a purified carbon dioxide stream that is provided to said step of providing a gaseous stream including greater than 1% by volume carbon dioxide.

28. A system for producing vinyl chloride, the system comprising:
(i) a first bioreactor including photosynthetic microorganisms that convert carbon dioxide to an organic intermediate, said first bioreactor having a carbon dioxide inlet and an outlet for the organic intermediate; and
(ii) a second bioreactor in fluid communication with the first bioreactor and including microorganisms that convert the organic intermediate produced in the first bioreactor to vinyl chloride, said second bioreactor having an outlet for gaseous materials including vinyl chloride, and said second bioreactor having an outlet for fluid materials including unreacted organic intermediate.

29. The system of claim 28, where the outlet for fluid materials of said second bioreactor is in fluid communication with said first bioreactor.

30. The system of claim 28, where the outlet for gaseous materials of said second bioreactor is in fluid communication with a carbon dioxide separator.

31. The system of claim 30, where the carbon dioxide separator includes an outlet for purified carbon dioxide, and where said outlet for purified carbon dioxide is in fluid communication with the first bioreactor.

32. The system of claim 31, further including a cryogenic distillation unit in fluid communication, either directly or indirectly, with said second bioreactor.

33. The system of claim 28, further including a dehydration unit disposed between and in fluid communication with said second bioreactor and said cryogenic distillation unit.
